(19) United States
(12) Patent Application Publication
(10) Pub. No.: US 2011/0159123 A1
(43) Pub. Date: Jun. 30, 2011

(54) Composition For Relieving Premenstrual Syndrome

(75) Inventor: Skyler Jesse Ditchfield, Ojai, CA (US)

(73) Assignee: Vescent Labs Inc.

(21) Appl. No.: 12/649,325

(22) Filed: Dec. 29, 2009

(51) Int. Cl. A61K 36/16; A61K 36/00; A61P 15/00

(52) U.S. Cl. 424/752; 424/775

(57) ABSTRACT

A composition is provided for relieving premenstrual syndrome. The composition comprises about 5% to about 95% by weight of a tribulus terrestris extract and about 5% to about 95% by weight of one or more herbal extracts selected from organic white willow extract, Red raspberry leaf extract, Chaste tree berry extract, Cramp bark extract, and Gingko biloba extract. The composition further comprises dehydroepiandrosterone, amino acids, vitamins and derivatives thereof, minerals and derivatives thereof, carbohydrates, citric acid, natural flavors, stevia, juice powder, maltodextrin, and silicon dioxide. The vitamins and derivatives thereof comprise, for example, vitamin A, vitamin B1, vitamin B2, vitamin B3, vitamin B6, vitamin B12, vitamin B9, vitamin B7, vitamin C, vitamin D3, and d-calcium pantothenate. The minerals comprise, for example, calcium, magnesium, manganese, sodium, and potassium. The ingredients are mixed in a liquid carrier and administered as a hot or cold beverage.

### BACKGROUND

Premenstrual syndrome (PMS) in women is a medical condition characterized by a number of both somatic and psychological symptoms. The most common somatic symptoms of PMS include, for example, swelling and tenderness of the breasts, abdominal pain, pelvic pain and cramps, feelings of bloatedness and weight gain, diminished activity, perspiration, skin lesions, edema, acne, etc. The most common psychological symptoms include irritability, agitation, anxiety, fatigue, mental depression, lethargy, insomnia or hypersomnia, etc.

The somatic and psychological symptoms of PMS are a major cause of suffering and discomfort to women, and cause substantial loss of time and efficiency in a women's life. The vast majority of available products including drugs such as antihistamines, nonsteroidal anti-inflammatories and diuretics, fail to provide adequate relief from the symptoms of PMS. Hence, there is a need for an easily administrable liquid composition useful for relieving PMS symptoms in women.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

The composition disclosed herein addresses the above stated need for an easily administrable composition that relieves symptoms of PMS in women. The composition comprises about 5% to about 95% by weight of a tribulus terrestris extract and about 5% to about 95% by weight of one or more herbal extracts selected from white willow extract, red raspberry leaf extract, chaste tree berry extract, cramp bark extract, and gingko biloba extract. In an embodiment, the herbal extracts are organic. In another embodiment, the composition comprises one or more components selected from white willow extract, red raspberry leaf extract, chaste tree berry extract, cramp bark extract, and gingko biloba extract. In yet another embodiment, the composition comprises one or more components selected from dehydroepiandrosterone, amino acids, vitamins and derivatives thereof, minerals and derivatives thereof, carbohydrates, citric acid, natural flavors, stevia, orange juice powder, maltodextrin, and silicon dioxide. The vitamins and derivatives thereof comprise, for example, vitamin A, vitamin B1, vitamin B2, vitamin B3, vitamin B6, vitamin B12, vitamin B9, vitamin B7, vitamin C, vitamin D3, and d-calcium pantothenate. The minerals comprise, for example, calcium, magnesium, manganese, sodium, and potassium. The ingredients are mixed in a liquid carrier and administered as a hot or cold beverage.

### BRIEF DESCRIPTION OF THE DRAWING

FIG. 1 exemplarily illustrates a table of ingredients used in the preparation of a beverage for relieving premenstrual syndrome.

### DETAILED DESCRIPTION OF THE INVENTION

A composition is provided for relieving premenstrual syndrome (PMS). As used herein, PMS refers to a combination of physical, psychological, and emotional symptoms related to a women's menstrual cycle. PMS symptoms include, for example, breast pain, emotional disturbance, cramping, hormonal imbalances, inflammation, etc. The composition for relieving PMS is mixed in water to obtain the PMS relieving beverage, which is ingested orally by the person seeking relief from PMS. In an embodiment, the beverage is an effervescent beverage.

As used herein, herbal extracts refer to extracts of Tribulus terrestris, white willow, red raspberry, chaste tree berry, cramp bark, and gingko biloba. The composition for relieving PMS symptoms comprises about 5% to about 95% by weight of a Tribulus terrestris extract and about 5% to about 95% by weight of one or more of a white willow extract, red raspberry leaf extract, chaste tree berry extract, cramp bark extract, and gingko biloba extract. The composition is mixed with about 2 to 10 ounces of cold or hot water, or a preferred liquid for ingestion and thereafter administered orally to a person. In an embodiment, the composition is effervescent. The composition is allowed to effervesce before ingestion.

In an embodiment, the composition for relieving PMS symptoms comprises one or more of a white willow extract, red raspberry leaf extract, chaste tree berry extract, cramp bark extract, and gingko biloba extract. In an embodiment, the herbal extracts are organic. The composition is mixed with about 2 to 10 ounces of hot water, cold water, or a preferred liquid for ingestion and thereafter administered orally to a person. In an embodiment, the composition is effervescent. The composition is allowed to effervesce before ingestion.

In an embodiment, the composition for relieving PMS symptoms comprises one or more of about 5% to about 65% by weight of white willow extract, about 5% to about 65% by weight of red raspberry leaf extract, about 5% to about 65% by weight of chaste tree berry extract, about 5% to about 65% by weight of cramp bark extract; and about 5% to about 80% by weight of gingko biloba extract.

Tribulus terrestris extract is obtained from the flowering plant Tribulus terrestris. Tribulus terrestris has a hormone balancing effect rendering it suitable for relieving the symptoms of PMS. White willow extract is obtained from willow trees and shrubs. White willow is known as a gentle natural pain reliever and has long lasting pain relieving effects. Red raspberry leaf extract is obtained from leaves of the raspberry plant. The red raspberry leaf is known as a uterine tonic. Red raspberry helps strengthen uterine walls and helps relieve menstrual cramping.

Chaste tree berry extract is obtained from the chaste tree. Chaste tree berry harbors hormone-balancing effects which is useful for a wide variety of menstrual disorders. The chaste tree berry relieves cramping and inflammation in the body and is a general tonic for building the endocrine system. Cramp bark extract is obtained from the cramp bark tree and is known for relieving menstrual cramping.

Gingko biloba extract is obtained from the gingko tree. Preliminary research has shown that Ginkgo biloba eases PMS symptoms such as breast discomfort and emotional shifts. One double-blind placebo-controlled study evaluated the benefits of Ginkgo biloba extract for women with PMS symptoms. The trial enrolled 143 women in the age group of 18 years to 45 years, and followed them for two menstrual cycles. The results showed that as compared to a placebo, Ginkgo biloba extract significantly relieved symptoms of PMS, especially breast pain and emotional disturbance.

In an embodiment, the compositions disclosed herein further comprises about 5% to about 50% by weight of dehydroepiandrosterone (DHEA). DHEA is a naturally occurring hormone. Research has shown promising results on the relief of PMS symptoms with DHEA.

In an embodiment, the compositions disclosed herein further comprises one or more amino acids selected from N-acetyl-tyrosine, L-tyrosine, and L-phenylalanine. N-acetyl-tyrosine and L-tyrosine are precursors of neurotransmitters, for example, L-dopa, dopamine, norepinephrine, and epinephrine. L-tyrosine is a non-essential amino acid that helps in reducing anxiety, depression, and headaches. L-phenylalanine is an electrically neutral amino acid. L-tyrosine and L-phenylalanine aid in the relief of depression and help women feel optimistic. Tyrosine stimulates the production of dopamine. Dopamine can be supplied as a medication that acts on the sympathetic nervous system. The composition further comprises an inhibitory neurotransmitter, for example, gamma amino butyric acid (GABA). Each hormone is involved in the regulation of two neurotransmitters, for example, serotonin and GABA. These brain chemicals have properties that protect against PMS symptoms. GABA is an amino acid that acts as a neurotransmitter to inhibit transmission of impulses from one nerve cell to another and plays a role in the stress response. GABA is known to elevate a woman's mood. The composition disclosed herein comprises about 5% to about 95% by weight of one or more amino acids or one or more amino acid derivatives.

In another embodiment, the composition for relieving the symptoms of PMS disclosed herein further comprises one or more of about 5% to about 90% by weight of one or more vitamins or derivatives of vitamins selected from vitamin A, vitamin B1, vitamin B2 (riboflavin), vitamin B3 (niacinamide), vitamin B6, vitamin B12, vitamin B9 (folic acid), vitamin B7 (biotin), vitamin C, vitamin D3, and d-calcium pantothenate. Vitamin A has been shown to reduce some of the PMS symptoms. Vitamin B12 reduces stress, prevents anemia, and is needed for all bodily functions. Vitamin B6 (pyridoxine) has also been called the woman's vitamin because of reported improvement in PMS. Vitamin B6 is one of the necessary ingredients required by the body to manufacture dopamine, one of the mood neurotransmitters. The other B vitamins are set in specific PMS doses. Vitamin D is the “sunshine” vitamin. Women who have diets rich in vitamin D have shown a reduction in PMS symptoms.

In another embodiment, the composition for relieving the symptoms of PMS disclosed herein further comprises about 5% to about 90% by weight of the composition of one or more minerals or mineral derivatives selected from calcium, magnesium, manganese, sodium, and potassium. Studies have suggested that manganese, in combination with calcium, reduces irritability, depression, and tension associated with PMS. A study suggests possible benefit from manganese in alleviating some PMS symptoms including anxiety, depression, irritability, and mood swings. Magnesium helps alleviate muscle and abdominal cramping and strengthens bones. When properly combined in a specific balance, these three key minerals work synergistically to help a woman's body relieve PMS symptoms.

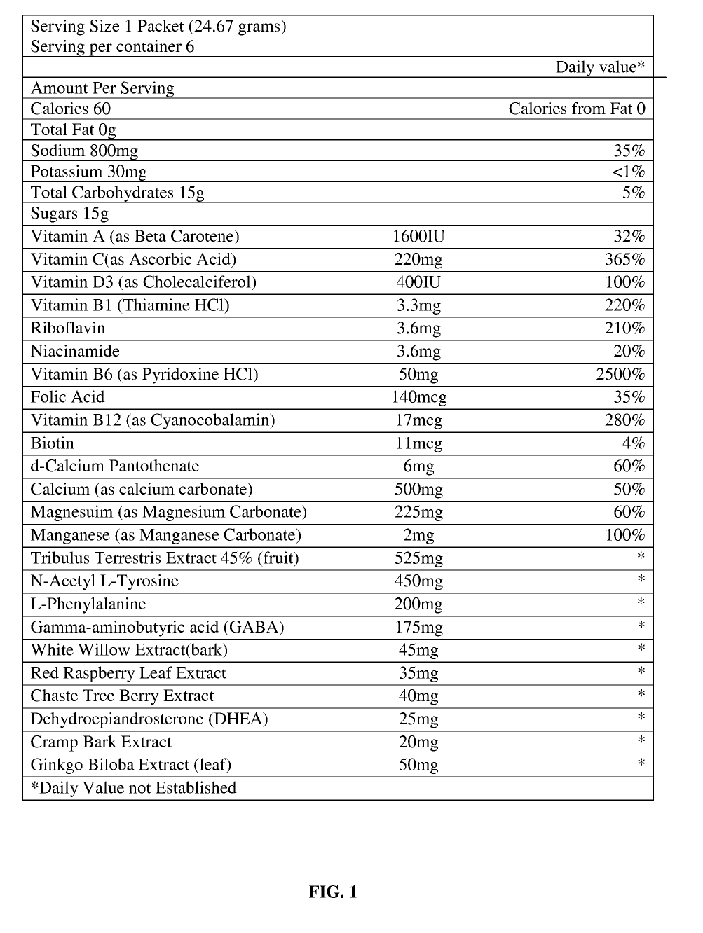

In another embodiment, the composition for relieving the symptoms of PMS disclosed herein further comprises about 5% to 85% by weight of the composition of one or more carbohydrates selected from monosaccharides, for example, fructose, and oligosaccharides, citric acid, natural flavors, stevia, orange juice powder, and maltodextrin. Fructose is a simple carbohydrate derived from fruit. Orange juice powder delivers a high dose of bioflavonoids. Doses of simple carbohydrates can work quickly to help alleviate some of the symptoms of PMS. Certain carbohydrates when ingested can increase the ratio of tryptophan to large neutral amino acids in the blood stream. This increase of the ratio of tryptophan to large neutral amino acids in the blood stream allows a higher level of tryptophan to enter the brain, which is necessary for increasing serotonin synthesis. A table of ingredients used in the preparation of a beverage for relieving PMS is exemplarily illustrated in FIG. 1.

In an embodiment, the composition for relieving the symptoms of relieving PMS comprises about 5% to about 50% by weight of tribulus terrestris extract; about 5% to about 50% by weight of cramp bark extract; about 5% to about 25% by weight of dehydroepiandrosterone; about 1% to about 25% by weight of vitamin B12; about 1% to about 15% by weight of vitamin D3; about 5% to about 50% by weight of vitamin B6; and 20% to about 60% by weight of a carbohydrate.

Exemplary compositions used in the preparation of the composition for relieving the symptoms of premenstrual syndrome are as follows:

### Example 1

A composition comprising 50% by weight of tribulus terrestris extract, 35% by weight of white willow extract and 15% of red raspberry leaf extract are mixed is mixed in water to prepare the PMS relieving beverage.

### Example 2

A composition comprising 35% by weight of white willow extract, 30% by weight of red raspberry leaf extract, and 35% by weight of chaste tree berry extract is mixed in water to prepare the PMS relieving beverage.

### Example 4

A composition comprising 40% by weight of cramp bark extract and 60% by weight of ginko biloba extract is mixed in water to prepare the PMS relieving beverage.

### Example 5

A composition comprising 80% by weight of tribulus terrestris extract, and 20% by weight of vitamin B6 is mixed in water to prepare the PMS beverage.

### Example 6

A composition comprising 50% by weight of tribulus terrestris extract, 25% by weight of calcium, and 25% by weight of magnesium is mixed in water to prepare the PMS relieving beverage.

### Example 7

A composition comprising 85% by weight of tribulus terrestris extract and 15% by weight of sodium bicarbonate is mixed in water to prepare the PMS relieving beverage.

### Example 8

A composition comprising 90% by weight of vitamin B6 and 10% by weight of manganese is mixed in water to prepare the PMS relieving beverage.

### Example 9

A composition comprising 10% by weight of tribulus terrestris extract, 5% by weight of cramp bark extract, 5% by weight of dehydroepiandrosterone, 5% by weight of vitamin B12, 5% by weight of vitamin D3, 5% by weight of vitamin B6, and 65% by weight of fructose is mixed in water to prepare the PMS relieving beverage.

### Example 10

A composition comprising 20% by weight of tribulus terrestris extract, 10% by weight of white willow extract, 10% by weight of cramp bark extract, 50% by weight of N-acetyl-tyrosine, and 10% by weight of L-phenylalanine is mixed in water to prepare the PMS relieving beverage.

### Example 11

A composition comprising 50% by weight of gamma amino butyric acid and 50% by weight of L-tyrosine is mixed in water to prepare the PMS relieving beverage.

### Example 12

A composition comprising 25% by weight of dehydroepiandrosterone and 75% by weight of fructose is mixed in water to prepare the PMS relieving beverage.

### Example 13

A composition comprising 25% by weight of tribulus terrestris extract, 25% by weight of cramp bark extract, 25% by weight of dehydroepiandrosterone, and 25% by weight of N-acetyl-tyrosine is mixed in water to prepare the PMS relieving beverage.

### Example 14

A composition comprising 20% by weight of tribulus terrestris extract, 20% by weight of potassium, 20% by weight of calcium, 20% by weight of magnesium, and 20% by weight of N-acetyl-tyrosine is mixed in water to prepare the PMS relieving beverage.

### Example 15

A composition comprising 50% by weight of tribulus terrestris extract, 10% by weight of N-acetyl-tyrosine, and 10% by weight of gamma amino butyric acid, 10% by weight of vitamin D3, and 20% by weight of vitamin B6, is mixed in water to prepare the PMS relieving beverage.

### Example 16

A composition comprising 95% by weight of tribulus terrestris extract and 5% by weight of manganese is mixed in water to prepare the PMS relieving beverage.

### Example 17

A composition comprising 40% by weight of cramp berry extract and 60% by weight of dehydroepiandrosterone is mixed in water to prepare the PMS relieving beverage.

### Example 18

A composition comprising 50% by weight of tribulus terrestris extract, and 50% by weight of calcium is mixed in water to obtain the PMS beverage.

### Example 19

A composition comprising 60% by weight of tribulus terrestris extract, 10% by weight of dehydroepiandrosterone, 15% by weight of N-acetyl-tyrosine, and 15% by weight of cramp bark extract, is mixed in water to prepare the PMS relieving beverage.

### Example 20

A composition comprising 15% by weight of tribulus terrestris extract, 5% by weight of cramp bark extract, 5% by weight of dehydroepiandrosterone, 5% by weight of vitamin B12, 5% by weight of vitamin D3, 5% by weight of vitamin B6, and 60% by weight of fructose is mixed in water to prepare the PMS relieving beverage.

### Example 21

A composition comprising about 5% to about 50% by weight of cramp bark extract; about 5% to about 25% by weight of dehydroepiandrosterone; about 1% to about 25% by weight of vitamin B12; about 1% to about 15% by weight of vitamin D3; about 5% to about 50% by weight of vitamin B6; and 20% to about 60% by weight of a carbohydrate.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention in its aspects.

I claim:

1. A composition for relieving premenstrual syndrome, comprising:
about 5% to about 95% by weight of tribulus terrestris extract, and
about 5% to about 95% by weight of one or more of white willow extract, red raspberry leaf extract, chaste tree berry extract, cramp bark extract, and gingko biloba extract.

2. The composition of claim 1, further comprising about 5% to about 50% by weight of dehydroepiandrosterone.

3. The composition of claim 1, further comprising about 5% to about 95% by weight of one of one or more amino acids and one or more amino acid derivatives.

4. The composition of claim 3, wherein the amino acids are one or more of N-acetyl-tyrosine, L-tyrosine and L-phenylalanine.

5. The composition of claim 1, further comprising about 10% to about 90% by weight of one of one or more vitamins and one or more derivatives of vitamins.

6. The composition of claim 5, wherein said one of said one or more vitamins and said one or more vitamin derivatives is one or more of vitamin A, vitamin B1, vitamin B2, vitamin B3, vitamin B6, vitamin B12, vitamin B9, vitamin B7, vitamin C, vitamin D3, and d-calcium pantothenate.

7. A composition for relieving premenstrual syndrome comprising one or more of white willow extract, red raspberry leaf extract, chaste tree berry extract, cramp bark extract, and gingko biloba extract.

8. The composition of claim 7 wherein:
said white willow extract is about 5% to about 65% by weight of the composition;
said red raspberry leaf extract is about 5% to about 65% by weight of the composition;
said chaste tree berry extract is about 5% to about 65% by weight of the composition;
said cramp bark extract is about 5% to about 65% by weight of the composition; and
said gingko biloba extract is about 30% to about 80% by weight of the composition.

9. The composition of claim 7 further comprising one or more of minerals and mineral derivatives.

10. The composition of claim 9, wherein the minerals comprise one of one or more of calcium, magnesium, manganese, sodium, and potassium.

11. The composition of claim 7, further comprising carbohydrates.

12. The composition of claim 7, further comprising one or more of citric acid, natural flavors, stevia, orange juice powder, maltodextrin.

13. A method of treating premenstrual syndrome by administering to a subject in need thereof a composition comprising:
about 5% to about 95% by weight of a tribulus terrestris extract; and
about 5% to about 95% by weight of one or more of white willow extract, red raspberry leaf extract, chaste tree berry extract, cramp bark extract, and gingko biloba extract.

14. The method of claim 13 wherein said composition further comprises about 10% to about 50% by weight of dehydroepiandrosterone.

15. The method of claim 13, wherein said composition further comprises about 5% to about 95% by weight of one of one or more amino acids and one or more amino acid derivatives.

16. The method of claim 15, wherein said one or more amino acids comprise N-acetyl-tyrosine, L-tyrosine, and L-phenylalanine.

17. A method of treating premenstrual syndrome by administering to a subject in need thereof a composition comprising one or more of white willow extract, red raspberry leaf extract, chaste tree berry extract, cramp bark extract, and gingko biloba extract.

18. The method of claim 17 wherein:
the white willow extract is about 5% to about 65% by weight of said composition;
the red raspberry leaf extract is about 5% to about 65% by weight of said composition;
the chaste tree berry extract is about 5% to about 65% by weight of said composition;
the cramp bark extract is about 20% to about 65%% by weight of said composition; and
the gingko biloba extract is about 30% to about 80% by weight of said composition.

19. The method of claim 17, wherein said composition further comprising one or more of:
about 10% to about 30% by weight of tribulus terrestris extract.
about 20% to about 60% by weight of N-acetyl-tyrosine; and
about 10% to about 30% by weight of L-phenylalanine.

20. A method of treating premenstrual syndrome by administering to a subject in need thereof a composition comprising one or more of:
about 15% to about 75% by weight of tribulus terrestris extract;
about 15% to about 65% by weight of cramp bark extract;
about 10% to about 50% by weight of dehydroepiandrosterone; and
about 15% to about 40% by weight of N-acetyl-tyrosine.

21. The method of claim 20, wherein said composition further comprises about 10% to about 90% by weight of one of one or more vitamins and one or more derivatives of vitamins.

22. The method of claim 20, wherein said composition further comprises carbohydrates.

23. The method of claim 20, wherein said composition further comprises one or more minerals, or one or more mineral derivatives, and wherein said one or more minerals comprise calcium, magnesium, manganese, sodium and potassium.
